(19) United States
(12) Patent Application Publication
(10) Pub. No.: US 2008/0031842 A1
(43) Pub. Date: Feb. 7, 2008

(54) Personal cleansing compositions

(76) Inventors: Dennis Eugene Kuhlman, Middletown, OH (US); Kenneth Eugene Kyte III, Lebanon, OH (US); Timothy Woodrow Coffindaffer, Maineville, OH (US); Matthew Donald McClure, Loveland, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/893,347

(22) Filed: Aug. 15, 2007

Related U.S. Application Data
(63) Continuation of application No. 10/797,981, filed on Mar. 11, 2004.

(51) Int. Cl. A61K 8/72; A61Q 19/10; A61Q 5/02

(52) U.S. Cl. 424/70.11

(57) ABSTRACT

The present invention is a lathering cleansing composition comprising an alkyl ethoxylated polymer, at least one lathering surfactant, an acrylate cross linked copolymer and a particulate material and a lathering surfactant. These compositions provide good lathering and are readily rinsed. The particulate materials enhance cleansing and exfoliation or provide conditioning befits without damage and, or irritation.

### CROSS REFERENCE TO RELATED APPLICATION

This application is a continuation of U.S. patent application Ser. No. 10/797,981, filed Mar. 11, 2004, pending.

### FIELD OF THE INVENTION

The present invention is for lathering, personal cleansing compositions providing superior cleansing with low irritation and good rinsing from skin, hair and the like.

### BACKGROUND OF THE INVENTION

Personal cleansing compositions are an important part of the daily hygienic regimen for a large group of both women and men throughout the world. It is, however, understood that certain cleansers, particularly facial cleansers, while good at removing sebum and grime from the skin and hair, may also cause insult in the form of skin irritation and, or damage to the surfaces being cleansed.

Formulators are challenged to reduce the irritation and damage caused by scrubs, while not compromising the formulations' ability to cleanse. To maintain sufficient cleansing action and/or exfoliation such formulations often include particulates, such as beads and encapsulated materials to replace harsh surfactants. Furthermore, in addition to cleansing, formulators are challenged to condition the hair and skin while minimizing damage and irritation. To accomplish this formulators suspend particles and, or droplets of materials such as oils or petrolatum to provide conditioning benefits. Particulates and oils droplets, however, must be suspended in these compositions in order to minimize migration (up or down) in the package they are sold in. While this is most easily accomplished in a thick, non-lathering composition, consumers often desire lathering products due to their spreading and rinsing characteristics while still in a mild surfactant base.

Suspending particulates such as beads, capsules and, or droplets of materials in these types of formulations is frequently accomplished through the use of suspension polymers. Suspension polymers known in the art for this purpose include acrylate cross-linked acid copolymers. Such materials are manufactured and sold by a number of suppliers including Noveon™, Inc. Noveon's sales literature discloses that addition of slight amounts of a polyol, such as glycerin or polyethylene glycol, may help reduce the “graininess” associated with gel compositions due to agglomeration of the polymer and, or compositional post-neutralization steps resulting in formation of salts; see “Polymer For Personal Care, Carbopol EDT® Resins: Formulation Tips”, March 1994.

Notwithstanding the benefits prescribed by Noveon™, use of such suspending agents in cleansing compositions can also impact the user's perception of rinsing the cleanser from the skin and hair. Through exhaustive consumer study, it is noted that when using these suspending polymers in cleanser compositions, consumers are left with the perception that the composition's lather poorly and, or do not rinse easily. In fact, it's not unusual for consumers to be left with the impression that these compositions feel slimy and, or do not rinse easily off after use.

It has also been noted that combinations of surfactants are also important for consumer satisfaction. Consumers appreciate surfactant systems that provide ample and rich lather yet are mild to the skin. High levels of amphoteric and/or zwitterionic surfactants relative to anionic surfactants are key for building mildness. High levels of amphoteric and/or zwitterionic surfactants, however, typically result in high levels of salt that negatively impacts the suspension capability of polymeric suspension systems. Absent such, many of these compositions are not commercially viable. Thus, there is a consumer demand for cleansing or scrub conditioning compositions that provide ample lather while being readily rinsed from the hair and skin.

### SUMMARY OF THE INVENTION

The present invention is a lathering cleansing composition comprising an alkyl ethoxylated polymer, at least one lathering surfactant, an acrylate cross linked copolymer, and a particulate material. These compositions provide good lathering and are readily rinse off. The particulate materials enhance cleansing and exfoliation as well as provide conditioning benefits without damage or irritation.

All percentages disclosed herein, unless otherwise stated, are by weight of the named material itself that is found in the compositions, thereby excluding for example the weight associated with carriers, impurities and by-products found in the raw material.

### DETAILED DESCRIPTION OF THE INVENTION

Alkyl Ethoxylated Polymers

The compositions of the present invention comprise alkyl ethoxylated polymers that are selected from the group consisting of di-alkyl, tri-alkyl- and combinations of di-alkyl and tri-alkyl substituted alkyl ethoxylated polymers. Alternatively mono-alkyl, di-alkyl, tri-alkyl, tetra-alkyl and all combinations thereof substituted alkyl ethoxylated polymers. The alkyl group can be saturated or unsaturated, branched or linear and contain a number of carbon atoms from about 12 carbon atoms to about 50 carbon atoms. The number of moles of ethylene oxide is greater than about greater than about 20, alternatively greater than about 40. These polymers are at levels from about 0.2% to about 1.0% alternatively from about 0.1% to about 2.0% and alternatively from about 0.05% to about 5.0%, of the composition.

The alkyl substitution of the alkyl ethoxylated polymer includes mono-alkyl, di-alkyl, tri-alkyl and tetra-alkyl substitution of the polymer and combinations thereof. Examples of the polymers that are mono alkyl substituted include: Steareth-100 available as Brij 700® from Uniqema Inc., Pareth alcohols available as Performathox 480® and 490® available from New Phase Technologies, Inc. The di-alkyl substituted polymers include PEG 120 methyl glucose dioleate available as Glutamate DOE-120® and Glucamate DOE-120® both from Chemron Corporation. The tri-alkyl substituted polymers include PEG 120 methyl glucose trioleate available as Glucamate LT® from Chemron Corporation. The tetra-alkyl substituted polymers include PEG 150 pentaerythrityl tetrastearate available as Crothix® from Croda Corporation. In the present invention, the usual substitution is di-alkyl, tri-alkyl or tetra-alkyl substitution of the polymer and combinations thereof. Even more usual are the di-alkyl, tri-alkyl substitution of the polymer and combinations thereof.

Cross-Linked Copolymers

The composition of the present invention includes cross-linked copolymers, are selected from the group consisting of cross-linked acid copolymers, cross-linked maleic anhydride copolymers and combinations thereof.

The cross-linked copolymers of the present invention are those typically used for thickening and suspending cosmetic agents in compositions such as shampoos, lotions and creams or other products having an aqueous electrolyte containing environment. The cross-linked copolymers are present in the present invention at levels from about 1% to about 2%, alternatively about 0.5% to about 3% and alternatively from about 0.1% to about 5% by weight of the composition.

The cross-linked acid copolymers in the present invention include alkyl substituted acid copolymers One class of alkyl substituted copolymers include a rheology modifying copolymer containing a cross-linked copolymer selected from the group consisting of unsaturated carboxylic acid, a hydrophobic monomer, a hydrophobic chain transfer agent, a cross linking agent, a steric stabilizers and combinations thereof. Carbopol EDT 2020™ from Noveon™ is an example of this suspending agent. Details regarding such suspending agents are found in U.S. Pat. No. 6,433,061, Marchant et al., Aug. 13, 2002.

Another class of copolymers includes a substantially cross-linked alkali-swellable acrylate copolymer described in U.S. Pat. No. 6,635,702. Carbopol Aqua SF-1™ from Noveon™ is an example of this type of suspending agent. Another class of commercially available copolymers useful herein include copolymers of C₁₀₋₃₀ alkyl acrylates with one or more monomers of acrylic acid, methacrylic acid, or one of their short chain (i.e. C₁₋₄ alcohol) esters, wherein the cross linking agent is an allyl ether of sucrose or pentaerytritol. These copolymers are known as acrylates/C₁₀₋₃₀ alkyl acrylate cross polymers and are commercially available as Carbopol® 1342, Pemulen® TR-1, and Pemulen® TR-2, from Noveon™. Yet another class of copolymers include polymers that fall under the description of acrylates/vinyl alkyl cross polymers, commercially available as Stabylen® 30 from 3V, Inc.

The cross-linked maleic anhydride copolymers include cross linked C₁-C₁₀ alkyl vinyl ether/maleic anhydride copolymers. Stabileze QM™ from ISP Corporation is an example of this type of material. In order to be effective, the maleic anhydride segment of this copolymer needs to be at least partially neutralized so that the copolymer becomes anionic.

Particularly useful are cross-linked cross copolymers include cross-linked alkyl substituted acid copolymers and alkali-swellable acrylate copolymers.

As described in Noveon's Technical Data Sheet #244, suspending capability of the formula can be approximated by yield value measurements. One method is the Brookfield yield value extrapolation method. The Brookfield Yield Value (herein BYV) is calculated by the following formula:

$$BYV ( dyn \text{/} {cm}^{2} ) = \frac{( {η}_{1} - {η}_{2} )}{100}$$

where, η₁ and η₂ are the apparent viscosities of the sample, measured at spindle speeds of 0.5 rpm and 1.0 rpm, respectively.

To determine the BYV for compositions having viscosities from about 5,000 cP to about 25,000 cP at 20 RPM, use a Brookfield viscometer with RV2 and RV3 spindles. As known by a person skilled in the art, in order to successfully suspend materials in the compositions, the BYV of such compositions varies depending on relationship of particulate size of the suspended material and the difference in densities of the dispersed phase and the continuous phase as expressed by Stokes Law. In the present invention, the BYV is greater than about 150 dyn/cm², alternatively greater than about 100 dyn/cm², alternatively greater than about 75 dyn/cm² and alternatively greater than about 50 dyn/cm².

Particulate Materials

Particulate materials for use in the present invention can generally be generally classified into one of two groups. These groups include: (1) cleaning or exfoliating agents and (2) conditioning agents.

The particulate cleansing or exfoliating agents can be derived from a wide variety of materials including those derived from inorganic, organic, natural, and synthetic sources.

The particulate cleansing or exfoliating agents of the present invention typically comprise from about 1% to about 5% alternatively from alternatively from about 0.5% to about 15% and alternatively from about 0.1% to about 30% by weight of the composition. Non-limiting examples of these materials include those selected from the group consisting of almond meal, alumina, aluminum oxide, aluminum silicate, apricot seed powder, attapulgite, barley flour, bismuth oxychloride, boron nitride, calcium carbonate, calcium phosphate, calcium pyrophosphate, calcium sulfate, cellulose, chalk, chitin, clay, corn cob meal, corn cob powder, corn flour, corn meal, corn starch, diatomaceous earth, dicalcium phosphate, dicalcium phosphate dihydrate, fullers earth, hydrated silica, hydroxyapatite, iron oxide, jojoba seed powder, kaolin, loofah, magnesium trisilicate, mica, microcrystalline cellulose, montmorillonite, oat bran, oat flour, oatmeal, peach pit powder, pecan shell powder, polybutylene, polyethylene, polyisobutylene, polymethylstyrene, polypropylene, polystyrene, polyurethane, nylon, teflon (i.e. polytetrafluoroethylene), polyhalogenated olefins, pumice rice bran, rye flour, sericite, silica, silk, sodium bicarbonate, sodium silicoaluminate, soy flour synthetic hectorite, talc, tin oxide, titanium dioxide, tricalcium phosphate, walnut shell powder, wheat bran, wheat flour, wheat starch, zirconium silicate, and mixtures thereof. Also useful are particles made from mixed polymers (e.g., copolymers, terpolymers, etc.), among such are polyethylene/polypropylene copolymer, polyethylene/pro-pylene/isobutylene copolymer, polyethylene/styrene copolymer, and mixtures thereof. Typically, the polymeric and mixed polymeric particles are treated via an oxidation process to destroy impurities and the like. The polymeric and mixed polymeric particles can also optionally be cross linked with a variety of common crosslinking agents, non-limiting examples including butadiene, divinyl benzene, methylenebisacrylamide, allyl ethers of sucrose, allyl ethers of pentaerythritol, and mixtures thereof. Other examples of useful particles include waxes and resins such as paraffins, carnuba wax, ozekerite wax, candellila wax, urea-formaldehyde resins, and the like. When such waxes and resins are used herein it is important that these materials are solids at ambient and skin temperatures.

Among the preferred water-insoluble, particulate materials useful herein are the synthetic polymeric particles and oils. Synthetic polymeric particles useful in the present invention are selected from the group consisting of polybutylene, polyethylene, polyisobutylene, polymethylstyrene, polypropylene, polystyrene, polyurethane, nylon, teflon, and mixtures thereof.

Oils suitable for use herein include any natural and synthetic materials with an overall solubility parameter less than about 12.5 (cal/cm³), preferably less than about 11.5 (cal/cm³).

By “overall solubility parameter” is meant that it is possible to use oils with higher solubility parameters than 12.5 (cal/cm³) if they are blended with other oils to reduce the overall solubility parameter of the oil mixture to less than about 12.5 (cal/cm³). For example, a small portion of diethylene glycol (sol par=13.61) could be blended with lanolin oil (sol par=7.3) and a co-solubilizing agent to create a mixture that has a solubility parameter of less than 12.5 cal/cm³.

Solubility parameters for the oils described herein are determined by methods well known in the chemical arts for establishing the relative polar character of a material. A description of solubility parameters and means for determining them are described by C. D. Vaughn, “Solubility Effects in Product, Package, Penetration and Preservation” 103 Cosmetics and Toiletries 47-69, October 1988; and C. D. Vaughn, “Using Solubility Parameters in Cosmetics Formulation”, 36 J. Soc. Cosmetic Chemists 319-333, September/October, 1988.

The conditioning particulate materials of the present invention typically comprise from about 2% to 15%, alternatively from about 1% to about 20% alternatively from alternatively from about 0.5% to about 30% and alternatively from about 0.1% to about 50% by weight of the composition. These oils include but are not limited to hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin and its derivatives, wax esters, beeswax derivatives, sterols and phospholipids, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof.

Non-limiting examples of silicone oils suitable for use herein include dimethicone copolyol, dimethylpolysiloxane, diethylpolysiloxane, mixed C₁-C₃₀ alkyl polysiloxanes, phenyl dimethicone, dimethiconol, and combinations thereof. Preferred are non-volatile silicones selected from dimethicone, dimethiconol, mixed C₁-C₃₀ alkyl polysiloxane, and combinations thereof. Non-limiting examples of silicone oils useful herein are described in U.S. Pat. No. 5,011,681 (Ciotti et al.).

Non-limiting examples of diglycerides and triglycerides suitable for use herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof. In addition any of the above oils that have been partially or fully hydrogenated are also suitable.

Non-limiting examples of acetoglyceride esters suitable for use herein include acetylated monoglycerides.

Non-limiting examples of alkyl esters suitable for use herein include isopropyl esters of fatty acids and long chain esters of long chain fatty acids, e.g. SEFA (sucrose esters of fatty acids). Lauryl pyrolidone carboxylic acid, pentaerythritol esters, aromatic mono, di or triesters, and cetyl ricinoleate are non-limiting examples of which include isopropyl palmitate, isopropyl myristate, cetyl ricinoleate and stearyl ricinoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use herein include oleyl myristate, oleyl stearate, oleyl oleate, and combinations thereof.

Non-limiting examples of lanolin and lanolin derivatives suitable for use herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol ricinoleate, hydroxylated lanolin, hydrogenated lanolin and combinations thereof.

Still other suitable oils include milk triglycerides (e.g., hydroxylated milk glyceride) and polyol fatty acid polyesters.

Still other suitable oils include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

The conditioning agents useful in the present invention are selected from the group consisting of droplets of emollient oils, skin care actives, vitamins, capsules and mixtures thereof. The capsules are generally made of gelatin, agar, or water-insoluble polymers and may contain emollient oils, vitamins, colored pigment, and additional ingredients, such as hair and skin actives as described below. The particle sizes of these capsules range from about 5 to about 3000 microns.

Lathering Surfactants

The articles of the present invention also comprise one or more lathering surfactants. A lathering surfactant defined herein as surfactant, which when combined with water and mechanically agitated generates a foam or lather. Preferably, these surfactants or combinations of surfactants should be mild, which means that these surfactants provide sufficient cleansing or detersive benefits but do not overly dry the skin or hair while still meeting the lathering criteria described above.

A wide variety of lathering surfactants are useful herein and include those selected from the group consisting of anionic lathering surfactants, nonionic lather surfactants, amphoteric lathering surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666, to Coffindaffer et al., issued Apr. 29, 1997. Cationic surfactants can also be used as optional components, provided they do not negatively impact the overall lathering characteristics of the required lathering surfactants

Concentrations of these surfactant are from about 10% to about 20%, alternatively from about 6% to about 25%, and alternatively from about 4% to about 30% by weight of the composition. To avoid skin irritation issues, the compositions should have a ratio by weight of the composition of anionic surfactant to amphoteric and/or zwitterionic surfactant is from about 1.1:1 to about 1:1.5, alternatively from about 1.25:1 to about 1:2, and alternatively from about 1.5:1 to about 1:3.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued December 30, 1975. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred. The alkoyl isethionates typically have the formula RCO—OCH₂CH₂SO₃M wherein R is alkyl or alkenyl, branched or linear of from about 10 to about 30 carbon atoms, preferably less than 20 carbon atoms, most preferably less than 18 carbon atoms and M is a water-soluble cation such as ammonium, sodium, potassium and triethanolamine. Non-limiting examples of these isethionates include those alkoyl isethionates selected from the group consisting of ammonium cocoyl isethionate, sodium cocoyl isethionate, sodium lauroyl isethionate, and mixtures thereof.

The alkyl and alkyl ether sulfates typically have the respective formulas ROSO₃M and RO(C₂H₄O)ₓSO₃M, wherein R is alkyl or alkenyl, branched or linear of from about 10 to about 30 carbon atoms, preferably less than 20 carbon atoms, most preferably less than 18 carbon atoms, x is from about 1 to about 10, and M is a water-soluble cation such as ammonium, sodium, potassium and triethanolamine. Another suitable class of anionic surfactants are the water-soluble salts of the organic, sulfuric acid reaction products of the general formula:
R1—SO₃-M
wherein R1 is chosen from the group consisting of a straight or branched chain, saturated aliphatic hydrocarbon radical having from about 8 to about 24, preferably about 10 to about 16, carbon atoms; and M is a cation. Still other anionic synthetic surfactants include the class designated as succinamates, olefin sulfonates having about 12 to about 24 carbon atoms, and b-alkyloxy alkane sulfonates. Examples of these materials are sodium lauryl sulfate and ammonium lauryl sulfate.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.). The fatty acids can also be synthetically prepared. Soaps are described in more detail in U.S. Pat. No. 4,557,853, cited above.

Other anionic materials include phosphates such as monoalkyl, dialkyl, and trialkylphosphate salts.

Other anionic materials include alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine), preferred examples of which are sodium lauroyl sarcosinate, sodium cocoyl sarcosinate, ammonium lauroyl sarcosinate, and sodium myristoyl sarcosinate. TEA salts of sarcosinates are also useful.

Also useful are taurates which are based on taurine, which is also known as 2-aminoethanesulfonic acid. Especially useful are taurates having carbon chains between C₈ and C₁₆. Examples of taurates include N-alkyltaurines such as the one prepared by reacting dodecylamine with sodium isethionate according to the teaching of U.S. Pat. No. 2,658,072, which is incorporated herein by reference in its entirety. Further non-limiting examples include ammonium, sodium, potassium and alkanolamine (e.g., triethanolamine) salts of lauroyl methyl taurate, myristoyl methyl taurate, and cocoyl methyl taurate.

Also useful are lactylates, especially those having carbon chains between C₈ and C₁₆. Non-limiting examples of lactylates include ammonium, sodium, potassium and alkanolamine (e.g., triethanolamine) salts of lauroyl lactylate, cocoyl lactylate, lauroyl lactylate, and caproyl lactylate.

Also useful herein as anionic surfactants are glutamates, especially those having carbon chains between C₈ and C₁₆. Non-limiting examples of glutamates include ammonium, sodium, potassium and alkanolamine (e.g., triethanolamine) salts of lauroyl glutamate, myristoyl glutamate, and cocoyl glutamate.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Suitable amphoteric or zwitterionic detersive surfactants for use in the compositions herein include those which are known for use in hair care or other personal care cleansing. Concentration of such amphoteric detersive surfactants are from about 1% to about 10%, alternatively from about 0.5% to about 20% by weight of the composition. Non-limiting examples of suitable zwitterionic or amphoteric surfactants are described in U.S. Pat. No. 5,104,646 (Bolich Jr. et al.) and U.S. Pat. No. 5,106,609 (Bolich, Jr. et al.).

Amphoteric detersive surfactants suitable for use in the compositions are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Preferred amphoteric detersive surfactants for use in the present invention are selected from the group consisting of cocoamphoacetate, cocoamphodiacetate, lauroamphoacetate, lauroamphodiacetate, and mixtures thereof.

Commercially available amphoteric surfactants include those sold under the trade names Miranol C2M Conc. N.P., Miranol C2M Conc. O.P., Miranol C2M SF, Miranol CM Special, Miranol Ultra (Rhodia, Inc.); Alkateric 2CIB (Alkaril Chemicals); Amphoterge W-2 (Lonza, Inc.); Monateric CDX-38, Monateric CSH-32 (Mona Industries); Rewoteric AM-2C (Rewo Chemical Group); and Schercoteric MS-2 (Scher Chemicals).

Zwitterionic detersive surfactants suitable for use herein include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Preferred zwitterionic detersive surfactants are the betaines and sulfobetaines, e.g., cocoamidopropylbetaine and cocoamidopropylhydroxysultaine.

Nonionic lathering surfactants for use in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety. Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Alkyl glucosides and alkyl polyglucosides are useful herein, and can be broadly defined as condensation articles of long chain alcohols, e.g. C₈₋₃₀ alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C₈₋₂₀ alkyl group, and n is an integer of from about 1 to about 9. Commercially available examples of these surfactants include decyl polyglucoside (available as APG 325 CS from Henkel) and lauryl polyglucoside (available as APG 600CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides, corresponding to the structural formula: 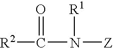
wherein: R¹ is H, C₁-C₄ alkyl, 2-hydroxyethyl, 2-hydroxy-propyl, preferably C₁-C₄ alkyl, more preferably methyl or ethyl, most preferably methyl; R² is C₅-C₃, alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polyhydroxy hydrocarbyl moiety having a linear hydrocarbyl chain with a least 3 hydroxyls directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G.B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety.

Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl)amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Non-limiting examples of preferred nonionic surfactants for use herein are those selected form the group consisting of C₈-C₁₈ glucose amides, C₈-C₁₈ alkyl polyglucosides, sucrose cocoate, sucrose laurate, lauramine oxide, cocoamine oxide, and mixtures thereof.

Preferred lathering surfactants for use herein are the following, wherein the anionic lathering surfactant is selected from the group consisting of ammonium lauroyl sarcosinate, sodium trideceth sulfate, sodium lauroyl sarcosinate, sodium myristoyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isethionate, sodium cetyl sulfate, sodium lauroyl lactylate, triethanolamine lauroyl lactylate, and mixtures thereof; wherein the nonionic lathering surfactant is selected from the group consisting of lauramine oxide, cocoamine oxide, decyl polyglucose, lauryl polyglucose, sucrose cocoate, C₁₂₋₁₄ glucosamides, sucrose laurate, and mixtures thereof; and wherein the amphoteric lathering surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

Lather Volume Test

The Average Lather Volume is a measurement determined by the Lather Volume Test. This test provides a consistent measurement of the volume of lather/foam generated by the articles described herein. The Lather Volume Test protocol is described as follows:
(1) wash hands thoroughly prior to the test using the product being tested. This will remove any soils which may affect the accuracy of the measurement;
(2) towel dry the hands;
(3) dispense, through a syringe, 1 cc of the composition in the palm of the hand;
(4) add 2 cc of water (medium hardness of about 8-10 grains per gallon) into the hands and rub hands together (very naturally) in a circular motion 5 times;
(5) rotate hand over hand 3 times, collecting the generated lather in a graduated cylinder or beaker big enough to hold the generated lather;
(6) level the lather with a plastic spatula and measure the volume; and
(7) complete the test a total of three times for each composition in order to derive the Average Lather Volume of the composition; calculating the Average Lather Volume by adding the measured lather volumes from each sample and dividing that number by three.

The compositions of the present invention preferably comprise a level of lathering surfactants wherein the Average Lather Volume of the composition is greater than or equal to about 15 ml, alternatively greater than or equal to about 20 ml, even more preferably greater than or equal to about 30 ml.

Additional Ingredients

The compositions of the present invention can contain a wide variety of ingredients including skin and hair care actives that are used in conventional product types, provided that they do not unacceptably alter the benefits of the invention. Additionally, these ingredients, when incorporated into the composition, should be suitable for use in contact with mammalian keratinous tissue without undue toxicity, incompatibility, instability, allergic response, and the like within the scope of sound judgment. The International Cosmetic Ingredient Dictionary and Handbook, 10^{th} Edition (2004) describes a wide variety of non-limiting cosmetic and pharmaceutical ingredients commonly used in the skin care industry, which are suitable for use in the compositions of the present invention. Examples of these and similar ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., salicylic acid), anti-wrinkle agents, anti-inflammatory agents, anti-atrophy agents, anti-caking agents, desquamation agents, antimicrobial and antifungal agents (e.g., methylchloroisothiazolinone/methylisothiazolinone, iodopropynyl butylcarbamate), antioxidants, retinoids, N-acylamino acid compounds, oil control agents (e.g., dehydroacetic acid or pharmaceutically acceptable salts) binders, biological additives, buffering agents, bulking agents, chelating agents, chemical additives, colorants, cosmetic astringents, cosmetic biocides, denaturants, drug astringents, emollients, external analgesics, film formers or materials, e.g., polymers, for aiding the film-forming properties or substantivity of the composition (e.g., copolymer of eicosene and vinyl pyrrolidone), humectants, opacifying agents, pH adjusters, propellants, reducing agents, sequestering agents, skin bleaching and lightening agents, skin-conditioning agents, skin firming agents, skin soothing and/or healing agents and derivatives, skin treating agents, surfactants, thickeners, amino sugars, and vitamins and derivatives thereof. Additional examples of suitable emulsifiers and surfactants can be found in, for example, U.S. Pat. No. 3,755,560, U.S. Pat. No. 4,421,769, and McCutcheon's Detergents and Emulsifiers, North American Edition, pages 317-324 (1986). It should be noted, however, that many materials may provide more than one benefit, or operate via more than one mode of action. Therefore, classifications herein are made for the sake of convenience and are not intended to limit the active to that particular application or applications listed.

### EXAMPLES

TABLE 1
Examples 1-8

| Ingredients | 1 | 2 | 3 | 4 | 5 | 6 | 7 | 8 |
|---|---|---|---|---|---|---|---|---|
| Acrylates Copolymer¹ | 1.50 | 1.50 | 2.0 | 2.0 | 2.0 | 1.25 | — | 1.50 |
| Acrylates/C₁₀₋₃₀ alkyl | — | — | — | — | — | — | 1.0 | — |
| acrylate crosspolymer² | | | | | | | | |
| Sodium Lauryl Sulfate | 2.0 | — | — | — | — | — | — | — |
| Sodium Laureth Sulfate | 8.0 | 3.0 | 6.0 | — | — | — | — | 3.0 |
| Ammonium Lauryl Sulfate | — | — | — | 6.0 | — | — | — | — |
| Sodium C₁₄₋₁₆ Olefin | — | — | — | — | 8.0 | — | — | — |
| Sulfonate | | | | | | | | |
| Sodium C₁₂₋₁₅ Pareth-9 | — | — | — | — | 2.0 | — | — | — |
| Sulfonate | | | | | | | | |
| Sodium Trideceth | — | — | — | — | — | 3.0 | 2.5 | — |
| Sulfate | | | | | | | | |
| Sodium Myristoyl | — | 5.0 | — | 2.0 | — | 3.0 | 2.5 | 5.0 |
| Sarcosinate | | | | | | | | |
| Sodium | — | 8.0 | — | — | — | 6.0 | 5.0 | 8.0 |
| Lauroamphoacetate³ | | | | | | | | |
| Sodium Hydroxide* | pH >6 | pH >6 | pH >6 | — | pH >6 | — | — | pH >6 |
| Triethanolamine* | — | — | — | pH >6 | — | — | pH 5.2 | — |
| Cocamidopropyl Betaine | 4.0 | — | 8.0 | 7.0 | 10.0 | — | — | — |
| Glycerin | 4.0 | 2.0 | 5.0 | 5.0 | 5.0 | 2.0 | 2.0 | 2.0 |
| Sorbitol | — | — | — | — | — | 2.0 | 2.0 | — |
| Salicylic Acid | — | — | — | — | 2.0 | 2.0 | 2.0 | — |
| Fragrance | 0.1 | 0.1 | 0.1 | 0.1 | 0.1 | 0.1 | 0.1 | 0.1 |
| Preservative | 0.3 | 0.3 | 0.3 | 0.3 | 0.3 | 0.15 | 0.15 | 0.3 |
| Jojoba Beads⁴ | 1.0 | — | — | — | — | — | — | — |
| Mica and Titanium | — | 0.2 | — | — | 0.2 | — | — | 0.2 |
| Dioxide⁵ | | | | | | | | |
| Mineral Oil, Vitamin | — | — | 1.0 | — | — | — | — | — |
| E, and Gelatin⁶ | | | | | | | | |
| Pumice⁷ | — | — | — | 1.0 | — | — | — | — |
| Oxidized | — | — | — | — | — | 2.0 | 2.0 | — |
| Polyethylene⁸ | | | | | | | | |
| PEG 120 Methyl | 0.5 | — | — | — | 0.5 | 0.25 | 0.25 | — |
| Glucose Trioleate⁹ | | | | | | | | |
| PEG 120 Methyl | — | 0.5 | 0.25 | — | — | — | — | 0.5 |
| Glucose Dioleate¹⁰ | | | | | | | | |
| Petrolatum, USP | — | — | — | — | — | — | — | 5.0 |
| PEG 150 Pentaerythrityl | — | — | — | 0.40 | — | — | — | — |
| Tetrastearate¹¹ | | | | | | | | |
| Citric Acid** | pH 5.5 | pH 5.5 | pH 5.5 | pH 5.5 | pH 4.5 | pH 5.5 | pH 5.5 | pH 5.5 |
| Water | QS to | QS to | QS to | QS to | QS to | QS to | QS to | QS to |
| | 100% | 100% | 100% | 100% | 100% | 100% | 100% | 100% |

*per the supplier use directions, the base is used to activate the acrylates copolymer
**acid can be added to adjust the formula to a lower pH
¹Carbopol Aqua SF-1 ® from Noveon ™, Inc.
²Carbopol Ultrez 21 ® from Noveon ™, Inc.
³Miranol ® Ultra L32 from Rhodia
⁴Florabeads ® from Floratech
⁵Pearl 3500 ® Engelhard
⁶Lipopearl ® from Lipo Chemicals, Inc.
⁷AEC Pumice ® from A & E Connock
⁸A-C ® Polyethylene from Honeywell/Accuscrub ® by Accutech
⁹Glucamate LT ® from Chemron
¹⁰Glucamate DOE-120 ® from Chemron
¹¹Crothix ® from Croda

Method of Making Examples 1-8:

Add Carbopol® to de-ionized free water of the formulation. Add all surfactants except cationics and betaines. If the pH is less than 6 then add a neutralizing agent (typically a base i.e., Triethanolamine, sodium hydroxide) to adjust to a pH greater than 6. If necessary, apply gentle heat to reduce viscosity and help minimize air entrapment. Add betaine and/or cationic surfactants. Add conditioning agents, additional rheology modifiers, pearlizing agents, encapsulated materials, exfoliants, preservatives, dyes, fragrances and other desirable ingredients. Lastly, if desired reduce the pH with an acid (i.e. citric acid) and increase viscosity by adding sodium chloride.

The methods of using the composition of the present invention include cleansing as well as cleansing and conditioning skin and, or hair.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A lathering personal cleansing composition comprising:
a. from about 0.05% to about 5.0% of an alkyl ethoxylated polymer;
b. from about 0.1% to about 5% of a cross-linked acid copolymer;
c. from about 0.1% to about 30% of a particulate material comprising conditioning agents; and
d. from about 4% to about 30% of a lathering surfactant selected from the group consisting of anionic, amphoteric, zwitterionic and mixtures thereof.

2. The composition according to claim 1 wherein the alkyl ethoxylated polymer is at a level from about 0.1% to about 2.0% of the composition.

3. The composition according to claim 1 wherein the alkyl substitution of the alkyl ethoxylated polymer is selected from the group consisting of mono-alkyl, di-alkyl, tri-alkyl, tetra-alkyl and all combinations thereof substituted alkyl ethoxylated polymers.

4. The composition according to claim 1 wherein the alkyl substitution of the alkyl ethoxylated polymer is selected from the group consisting of di-alkyl, tri-alkyl- and combinations of di-alkyl and tri-alkyl substituted alkyl ethoxylated polymers.

5. The composition of claim 3 wherein the alkyl group is selected from the group consisting of saturated, unsaturated, branched, linear alkyl groups and combinations thereof having from about 12 to about 50 carbon atoms.

6. The composition of claim 4 wherein the number of moles of ethylene oxide is greater than about 40.

7. The composition of claim 1 wherein the cross linked copolymer is selected from the group consisting of cross-linked acid copolymers, cross-linked maleic anhydride copolymers and mixtures thereof.

8. The composition according to claim 6 wherein the cross-linked acid copolymers is an alkyl substituted copolymer containing a cross-linked copolymer selected from the group consisting of unsaturated carboxylic acid, a hydrophobic monomer, a hydrophobic chain transfer agent, a cross linking agent, a steric stabilizers and combinations thereof.

9. The composition according to claim 1 wherein the cross linked copolymers are alkali-swellable acrylate copolymers.

10. The composition according to claim 1 wherein the conditioning agent is selected from the group consisting of droplets of emollient oils, skin care actives, vitamins, capsules containing these materials and mixtures thereof.

11. The composition according to claim 10 wherein the capsules are in the range of about 5 to about 3000 microns.

12. The composition according to claim 1 wherein the lathering surfactant is at a level wherein the Average Lather Volume of the composition is greater than or equal to about 15 ml.

13. The composition according to claim 1 wherein the lathering surfactant is at a level from about 6% to about 25% by weight of the composition.

14. The composition according to claim 1 wherein the surfactants have a ratio of anionic surfactant to amphoteric lathering and zwitterionic lathering surfactants from about 1.5:1 to about 1:3.

15. The composition according to claim 1 wherein the anionic lathering surfactants are selected from the group consisting of sarcosinates, sulfates, sulfonates, isethionates, taurates, phosphates, lactylates, glutamates, and mixtures thereof.

16. The composition according to claim 1 wherein the composition has a BYV greater than about 50 dyn/cm².

17. The composition according to claim 1, wherein said amphoteric surfactant comprises 1% to about 10%, by weight of the personal care composition.
